(19) United States
(12) Patent Application Publication
(10) Pub. No.: US 2022/0142531 A1
(43) Pub. Date: May 12, 2022

(54) DEVICE AND METHOD FOR MEASURING BLOOD CONSTITUENTS

(71) Applicant: Shani Biotechnologies LLC, Austin, TX (US)

(72) Inventor: Sanjay Gokhale, Arlington, TX (US)

(73) Assignee: Shani Biotechnologies LLC, Austin, TX (US)

(21) Appl. No.: 17/581,415

(22) Filed: Jan. 21, 2022

Related U.S. Application Data
(63) Continuation-in-part of application No. 17/542,765, filed on Dec. 6, 2021.
Application No. 17/542,765 is a division of application No. 17/317,171, filed on May 11, 2021, now Pat. No. 11,191,460.
(60) Provisional application No. 63/072,504, filed on Aug. 31, 2020.
(60) Provisional application No. 63/052,115, filed on Jul. 15, 2020.

(51) Int. Cl. A61B 5/1455; A61B 5/00; A61B 5/103

(52) U.S. Cl. CPC A61B 5/14551; A61B 5/443; A61B 5/7435; A61B 5/748; A61B 5/1032

(57) ABSTRACT

The present disclosure is directed to a method and device to determine a value of a blood constituent of a mammal. The device includes a plurality of light emitting diodes, one or more sensors and a processor.

### CROSS REFERENCE TO RELATED APPLICATIONS

This application is a continuation-in-part of co-pending application Ser. No. 17/542,765, filed Dec. 6, 2021, which is a divisional application of co-pending application er. No. 17/317,171, filed on May 11, 2021, now U.S. Pat. No. 11,191,460, which claims the benefit of both of U.S. Provisional Patent Application Ser. No. 63/052,115 filed on Jul. 15, 2020 and U.S. Provisional Patent Application Ser. No. 63/072,504 filed on Aug. 31, 2020, the entire contents of each of which are herein incorporated by reference.

### BACKGROUND OF THE DISCLOSURE

The measurement and monitoring of blood constituents such as hemoglobin (Hb) and its species such as oxy-hemoglobin (O-Hb), creatinine, glucose, lactate, and others, is often an invasive procedure. The blood draw can cause discomfort to the patient and increases the risk of infection. The adverse effects of constant and often difficult blood draws on newborns and infants are even more pronounced. Frequent and constant blood constituent measurements are clinically indicated in a wide variety of conditions such as (but not limited to) acute and/or chronic kidney failure, hemodialysis status, sepsis, shock, malignancies, anemia, emergency and critical care including patients infected with Coronavirus Disease of 2019 (COVID-19), hemoglobinopathies such as sickle cell disease (hemoglobin-S), hematological malignancies, and peri-operative management after major surgeries such as spine surgery etc. In addition, these parameters are part of routine health maintenance in healthy subjects and are increasingly being used to monitor athletic performance.

Thus, frequent measurements of blood constituents have wide applications in healthy adults and children, athletes, patients recovering from various illnesses, as well as patients admitted in the hospital setting. Although the available invasive techniques are established, they require the presence of medical personnel and laboratory equipment. These requirements drive up the cost, as well as the amount of time for the results to be available for the clinical use.

The measurement and monitoring of blood constituents such as creatinine is typically an invasive procedure with a blood draw, which can cause discomfort to the patient and increases the risk of infection. The adverse effects of constant and often difficult blood draws on newborns and infants are even more pronounced. Frequent and constant creatinine measurements are clinically indicated in a wide variety of conditions such as acute and chronic kidney failure, patients on hemodialysis, emergency and critical care including patients infected with COVID-19, sepsis or shock, malignancies, and peri-operative management after major surgeries such as spine surgery etc.

Thus, frequent measurements of creatinine have wide applications in healthy adults and children, athletes, patients recovering from various illnesses, as well as patients admitted in the hospital setting. So, as said earlier although the available invasive techniques are established, they require the presence of medical personnel and laboratory equipment. These requirements drive up the cost, as well as the amount of time for the results to be available for the clinical use.

Additionally, measurement of blood constituents such as glucose have wide implications. Blood glucose in the bloodstream supplies energy at the cellular level for mammals. Blood glucose levels change throughout the day. After eating food, glucose levels typically rise and then settle after about an hour. Glucose levels are typically at their lowest point before the first meal of the day. Insulin acts as a key hormone controller of the blood glucose levels. Lack of insulin or insulin-resistance leads to impaired ability to handle glucose, leading to high blood glucose levels (hyperglycemia) and diabetes mellitus. On the other hand, extreme exercise and sickness can increase the body's energy demand, and lead to dangerously low levels of blood glucose (hypoglycemia). Thus, it is important to maintain the blood glucose level in the physiological range for optimal health.

Recent estimates indicate there were 171 million people in the world with diabetes in the year 2000 and this is projected to increase to 366 million by 2030. In the U.S.A. alone, 34.2 million people have diabetes (˜10.5% of the US population).

Diabetes is a condition primarily defined by the level of hyperglycemia giving rise to risk of microvascular damage (retinopathy, nephropathy, and neuropathy). It is associated with reduced life expectancy, significant morbidity due to specific diabetes related microvascular complications, increased risk of macrovascular complications (ischemic heart disease, stroke, and peripheral vascular disease), and diminished quality of life. The American Diabetes Association (ADA) estimated the national costs of diabetes in the USA for 2002 to be $ 132 billion, increasing to $ ˜192 billion in 2020.

People with diabetes must regularly check their blood glucose levels to know how much medication to use, or to keep track of fluctuating levels. This monitoring is generally done at home using a finger prick blood test. Although accurate, this test can be inconvenient, and there are concerns that many patients are not testing themselves as frequently as they should.

Currently, there are several methods for blood glucose estimations.

1) Venous blood collection and laboratory analysis is the traditional, standard method. This method requires specialized equipment and personnel and is not feasible for frequent at home or point of care glucose monitoring.

2) Glucometer is the most used, point of care blood glucose estimation method. It requires finger prick and obtaining blood sample for application on the test strips. Most glucose meters are based on electrochemical technology, they use electrochemical test strips to perform the measurement. The glucometer estimated glucose values have poor validity and reliability. Additionally, glucometer measures capillary glucose levels. The capillary blood glucose value is typically different than the serum blood glucose value. Since almost all the clinical decisions and guidelines are based on serum blood glucose levels, measurement of capillary blood glucose might not provide the clinically required accurate value, affecting the clinical decisions (such as to administer insulin).

3) Other methods include various sensors, such as temperature, ultrasound and/or magnetic sensors to estimate blood glucose levels. Alternatively, implantable glucose sensors can also be used for long term measurements.

Additionally, measurement of blood constituents such as lactate have wide implications. Lactate is a substance produced during metabolism in the muscles and other parts of the body. Normal blood lactate levels are between 0.6-2.4 mmol/L. Typical measurement of lactate is an invasive, blood draw procedure, which can cause discomfort and increases the risk of infection. The adverse effects of constant and often difficult blood draws on newborns and infants are even more pronounced. Elevated lactate levels above normal range suggest a health condition. Frequent and constant blood lactate measurements are done for patients with trauma, major surgeries, and severe infections such as sepsis. Lactate is an important blood biomarker in patients suffering from Diabetes Mellitus, kidney diseases and drug overdose or poisoning as well. In addition, lactate is a part of routine health maintenance in healthy subjects and are increasingly being used to monitor athletic performance.

Although the available invasive techniques are established, they require the presence of medical personnel and laboratory equipment. These requirements drive up the cost, as well as the amount of time for the results to be available for the clinical use. There are a few ‘point of care (POC)’ Lactate tests, which can be done in the field or potentially at a bedside, however, these tests require obtaining a blood sample from the patient. Furthermore, the accuracy of such methods is not well established.

What is desired is a non-invasive system, devices, and method to measure various blood components. Embodiments of the present disclosure provide devices and methods that address the above needs.

### SUMMARY OF THE DISCLOSURE

The present disclosure is directed to the measurement of blood component concentration using reflectance spectroscopy. The advantages of this technology are a simple, portable, and easy to use hand-held device (as described below) to measure the blood component concentration in real time, and a method for continuous (or nearly continuous) non-invasive blood component monitoring.

### BRIEF DESCRIPTION OF THE DRAWINGS

The present disclosure will be better understood by reference to the following drawings, which are provided as illustrative of certain embodiments of the subject application, and not meant to limit the scope of the present disclosure.

FIG. 1 is a graphical illustration of a device of the present disclosure.

FIG. 2 is a graphical illustration of a probe of the device of the present disclosure.

FIG. 3 is a graphical illustration of a probe of the device and a housing of the present disclosure.

FIG. 4 is a graphical illustration of a probe near a human mammal's skin surface.

FIG. 5 is a graphical illustration of a probe near a human mammal's skin surface.

FIG. 6 is a graph of the reflection of light level as compared to hemoglobin level.

FIG. 7 is a graph of the reflection of light level as compared to hemoglobin level.

FIG. 8 is a graph of the reflection of light level as compared to hemoglobin level.

FIG. 9 is a graph of light signal measurements over four different time points for four different areas of a human mammal's skin surface in the same mammal.

FIG. 10 is a graph of absorption percentage over varying wavelengths for two types of hemoglobin: fetal hemoglobin (Hb-F) and adult hemoglobin (Hb-A).

FIG. 11 is a graph of the reflection of light level as compared to hemoglobin level.

FIG. 12 is a graph of the R value as compared to various skin colors; and

FIG. 13 is a graph of E1 to E2 ratio over time.

FIG. 14 is a graph of creatinine level vs reflectance ratio.

FIG. 15 is a graph of the reflection of light level as compared to the glucose level of a subject.

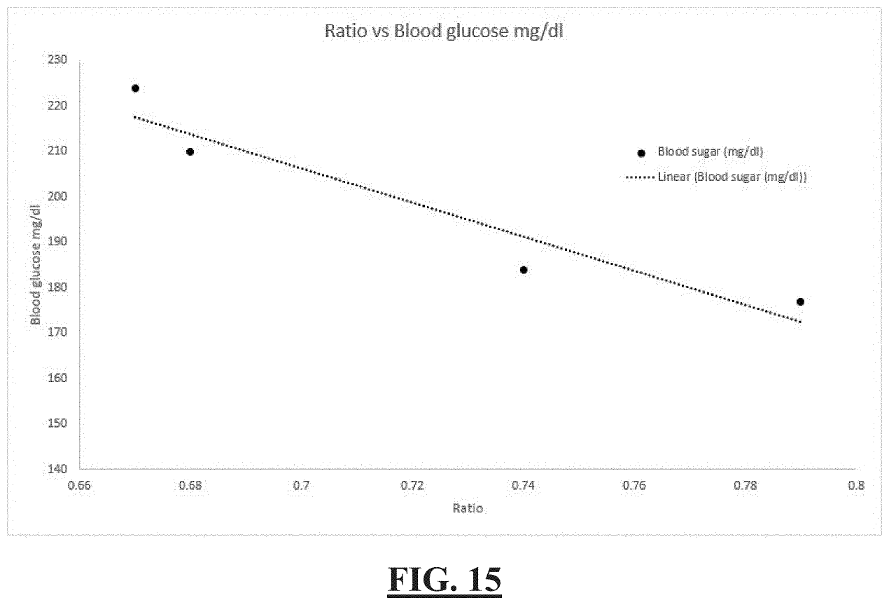

FIG. 16A is a graph of the reflection of light level as compared to the glucose level of two subjects.

FIG. 16B is a graph of glucose level as compared to ratios for several data points.

FIGS. 17A-17C are circuit diagrams of a sensor of the disclosure.

FIG. 18 is a graph of the reflection of light level as compared to lactate level.

FIG. 19 is a graph of the reflection of light level as compared to lactate level before and after exercise, measured with the disclosed device and a blood draw method.

### DETAILED DESCRIPTION OF THE DISCLOSURE

In the discussion and claims herein, the term “about” indicates that the value listed may be somewhat altered, as long as the alteration does not result in nonconformance of the process or device. For example, for some elements the term “about” can refer to a variation of ±0.1%, for other elements, the term “about” can refer to a variation of ±1% or ±10%, or any point therein.

As used herein, the term “substantially”, or “substantial”, is a broad term and is used in its ordinary sense, including, without limitation, being largely but not necessarily wholly that which is specified, which is equally applicable when used in a negative connotation to refer to the complete or near complete lack of an action, characteristic, property, state, structure, item, or result. For example, a surface that is “substantially” flat would mean either completely flat, or so nearly flat that the effect would be the same as if it were completely flat.

As used herein terms such as “a”, “an” and “the” are not intended to refer to only a singular entity but include the general class of which a specific example may be used for illustration.

As used herein, terms defined in the singular are intended to include those terms defined in the plural and vice versa.

References in the specification to “one embodiment”, “certain embodiments”, some embodiments” or “an embodiment”, indicate that the embodiment(s) described may include a particular feature or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

For purposes of the description hereinafter, the terms “upper”, “lower”, “right”, “left”, “vertical”, “horizontal”, “top”, “bottom”, and derivatives thereof shall relate to the invention, as it is oriented in the drawing figures. The terms “overlying”, “atop”, “positioned on” or “positioned atop” means that a first element, is present on a second element, wherein intervening elements interface between the first element and the second element. The term “direct contact” or “attached to” means that a first element and a second element are connected without any intermediary element at the interface of the two elements.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc.

The present disclosure is directed to devices and methods of measuring various blood components, such as different forms of hemoglobin, such as oxy-hemoglobin (O-Hb) and deoxy-hemoglobin (d-Hb).

A hemoglobin molecule has two units, Heme and Globin. The Heme part has one iron as Fe⁺² or Ferrous form and Pyrrole rings. Globin is tetrameric or with four amino acid chains. These chains are Alpha, Beta, Gamma and Delta globin chains. Hemoglobin therefore has a Heme and Four Globin Chains. For example, Hb-A has two alpha and two beta chains. Table 1 below summarizes the three types of Hemoglobins in health.

TABLE 1

| | | | |
|---|---|---|---|
| Hemoglobin-A | Heme + α2β2 | Hb-A | 98 to 98.5% of |
| | | | total hemoglobin |
| Hemoglobin-A2 | Heme + α2δ2 | Hb-A2 | 1 to 1.5% of |
| | | | total hemoglobin |
| Hemoglobin-F | Heme + α2γ2 | Hb-F | In Newborns till |
| | | | around 8 months |

Oxy-hemoglobin refers to the amount of hemoglobin having oxygen bound to the heme component, while deoxy-hemoglobin refers to the amount of hemoglobin not having bound oxygen. Oxy-hemoglobin and total hemoglobin retain a ratio based on health. When hemoglobin is broken down, globin chains are added to the amino acid pool. Heme is split and all the Pyrrole rings open. These are later metabolized into Bilirubin. When the concentrations are high, more light is being absorbed. Hence, by measuring reflected light, as discussed further below, the concentration of oxy-hemoglobin, and deoxy-hemoglobin can be determined.

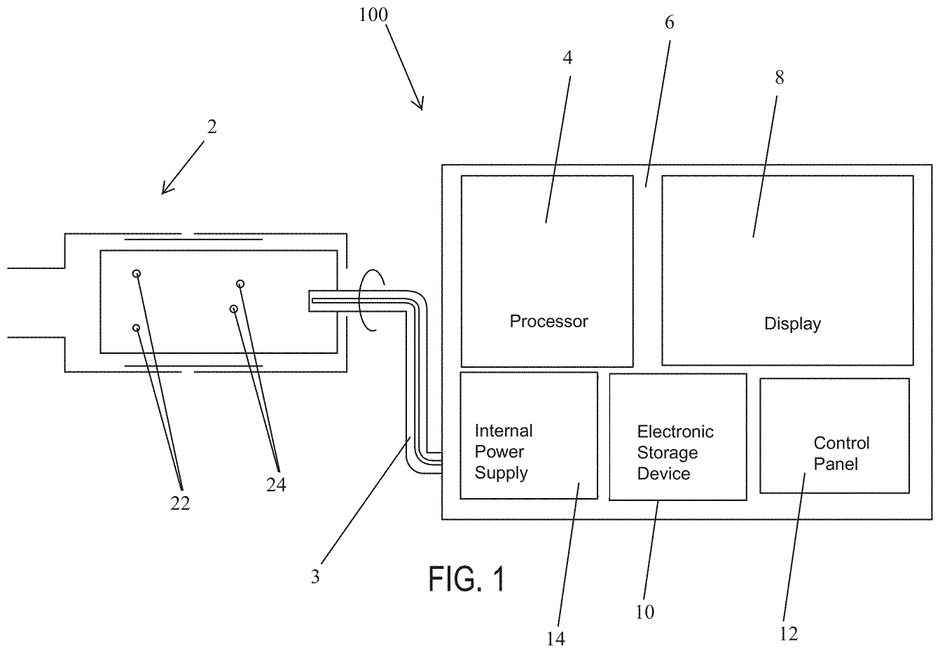

Based on the above a probe device can be utilized to measure reflected light to determine a ratio of oxy-hemoglobin to deoxy-hemoglobin. For example, a probe device 100 is shown in FIG. 1. Oxy-hemoglobin (O-Hb) and deoxy-hemoglobin (d-Hb) have unique spectroscopic properties as compared to each other. O-Hb has an absorption peak between about 515 nm to about 535 nm, between about 520 nm to about 530 nm, or about 525 nm. d-Hb has an absorption peak at between about 540 nm to about 560 nm, between about 545 nm to about 555 nm, or about 550 nm.

The device 100 can include a probe (device) 2 and a processor 4. As used herein, the term “processor” may refer to, is part of, or includes circuitry capable of sequentially and automatically carrying out a sequence of arithmetic or logical operations; recording, storing, and/or transferring digital data. The term “processor” may refer to one or more application processors, one or more baseband processors, a physical central processing unit (CPU), a single or multiple-core processor, and/or any other device capable of executing or otherwise operating computer-executable instructions, such as program code, software modules, and/or functional processes.

In this embodiment the probe 2 is connected to the processor 4 through a suitable cable 3, which is configured to transmit electrical signals. However, in other embodiments, the probe 2 can wirelessly communicate with the processor 4 through any suitable wireless protocol, including but not limited to Wi-Fi, Bluetooth®, Near Field Communication (NFC), etc. In yet other embodiments, the processor 4 can be within the probe 2 itself.

The processor 4 can be included in a housing 6. The housing 6 can also include an electronic storage device 10. As used herein, the term “electronic storage device” includes any type of integrated circuit, microcontroller and/or other storage device adapted for storing digital data including, without limitation, ROM, PROM, EEPROM, DRAM, SDRAM, DDR/2 SDRAM, EDO/FPMS, RLDRAM, SRAM, “flash” memory (e.g., NAND/NOR), 3D memory, and PSRAM.

The housing 6 can also include a display 8. The display 8 can be any suitable display, such as a liquid crystal display (LCD), a cathode ray tube display, a light emitting diode (LED) display, or the like, which can display various information determined by the probe 2 and/or stored within the memory 10. Optionally, the display 8 can also be an input by receiving touch input from a user on or near a portion of the display 8. Alternatively, or in addition to, the display 8 being an input, the housing 6 can also include a control panel 12, which can accept various inputs from a user. These inputs are described in more detail below.

Optionally, in this embodiment, the housing 6 can also include an internal power supply 14, such as a battery. However, in other embodiments, the probe 2 and/or processor 4 can receive power from an external source. In yet other embodiments, the probe 2 itself can include a power supply 14.

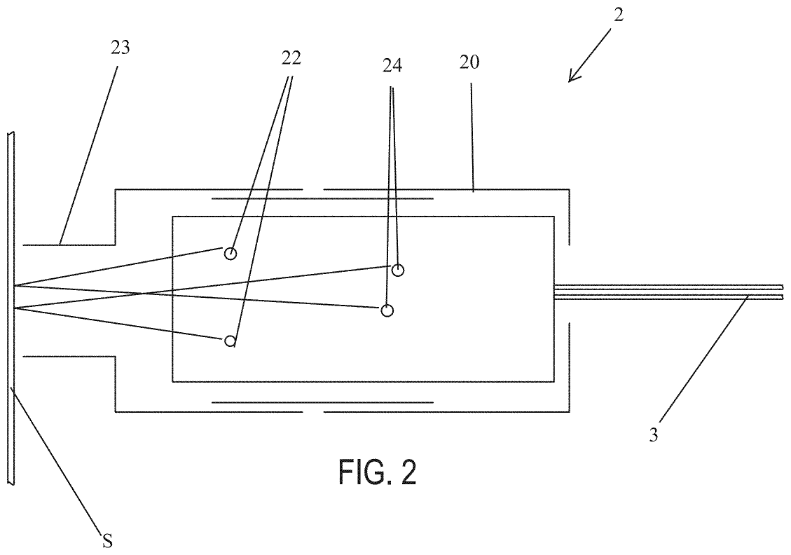

The probe 2 is illustrated in more detail in FIG. 2, including a nozzle 23 of the probe 2. The nozzle 23 can be any suitable, hollow, or tubular structure that can be formed to be any suitable length to allow for accurate measurement of reflected light by the probe 2.

In FIG. 2, within the probe housing 20 are a plurality of light emitting diodes 22. The wavelengths of each of the plurality of these light emitting diodes 22 can be the same or different, can be fixed or variable, and can be any suitable wavelength, in any suitable range, such as about 450 nm to about 580 nm. Specific examples of such wavelengths include, but are not limited to about 525 nm, about, 545 nm, about 550 nm, and about 575 nm. However, in other embodiments, the wavelengths can differ from any of the above values by about 0.001%, about 0.01%, about 0.1%, about 0.5%, about 1%, about 2%, about 3%, about 4%, about 5%, about 7%, about 9%, about 10%, about 13%, about 15%, about 20% or more.

The plurality of light emitting diodes 22 can be activated individually, or in any suitable sequence, to illuminate a surface, such as a skin surface (any suitable portion of the epidermis) of a mammal.

Upon illumination from the light emitting diodes 22, one or more sensors 24 (two are shown in the figures for illustrative purposes), which are configured to sense an amount of light, for example a photodiode, do measure the light reflected from the surface. These one or more sensors 24 then convert that measured light to a suitable electrical signal. In the present disclosure, the light emitting diodes 22 can emit light at any given interval, from about 0.1 second or less, to about 0.5 seconds or more, about 1 second or more, about 5 seconds or more, about 30 seconds or more, about 1 minute or more, about 2 minutes or more, about 5 minutes or more, or about 10 minutes or more. Accordingly, the one or more sensors 24 can be configured to sense the amount of reflected light at times corresponding to whatever time period is selected for light emission.

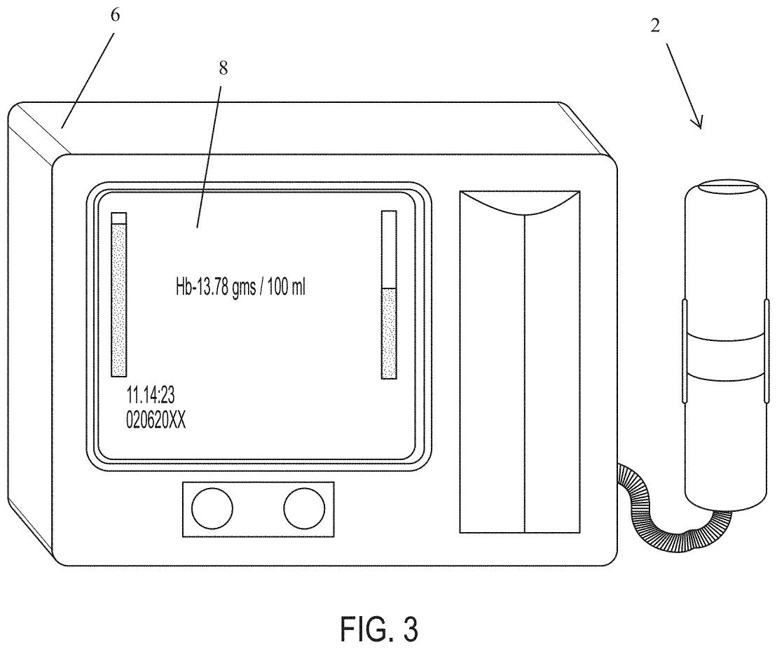

FIG. 3 is one embodiment of a probe 2 connected to a housing 6, with the housing 6 including the display 8 and various inputs and controls. The opening at the vertical upper portion of the probe 2 is where each light is transmitted (from the plurality of light emitting diodes 22) and collected (by the one or more sensors 24) through. This opening can be a void, or can include a substantially transparent barrier, such as a plastic and/or glass barrier.

The display 8, which can optionally be used as an input, can be used to display various data, such as the name of the mammal the probe 2 is to be applied to, date, time, status of the probe 2, power indicator, total hemoglobin, oxy-hemoglobin level, deoxy-hemoglobin level, and/or a ratio of oxy-hemoglobin to deoxy-hemoglobin.

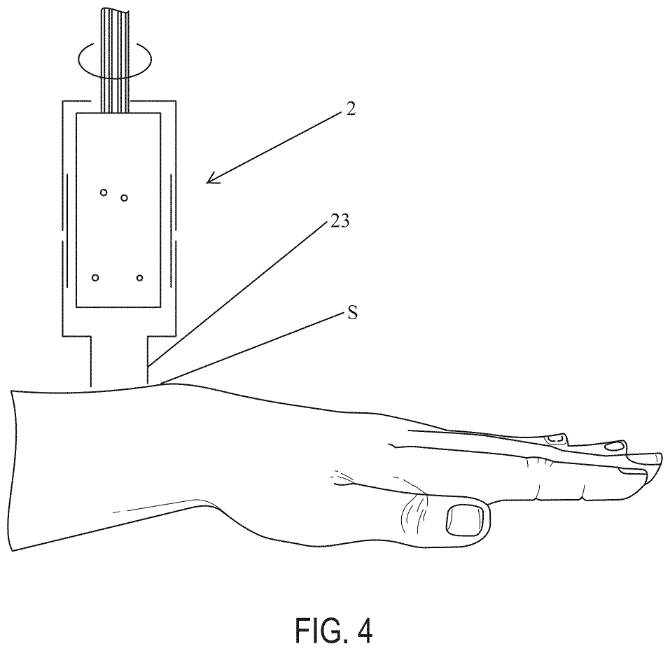

The probe 2 can be placed into contact with any portion of a mammal's skin (S), for example a human's wrist and/or hand as shown in FIG. 4. As can be seen in FIG. 4, the nozzle 23 of the probe 2 is placed near or in contact with the portion of the human's wrist and is held there by another user (or the human themselves, or by a wearable structure).

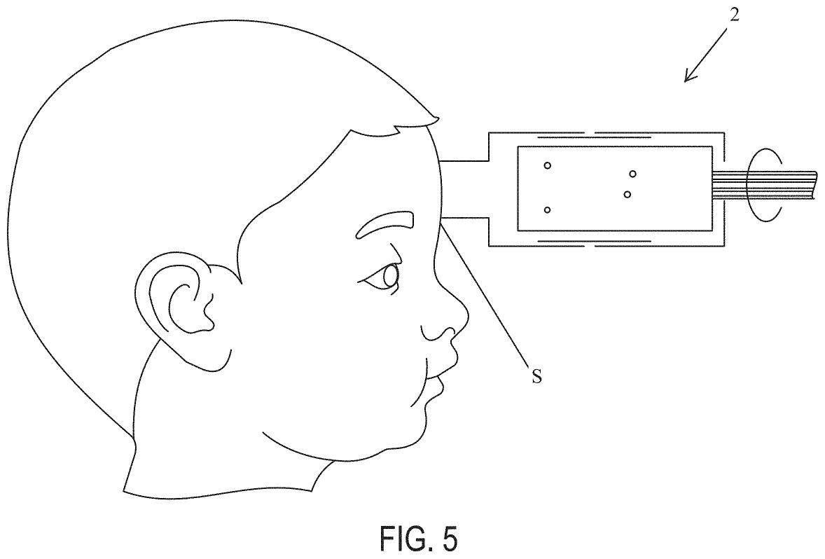

In another embodiment, the probe can be placed into contact with a human's forehead, as seen in FIG. 5.

Alternatively, to the probe of FIGS. 4 and 5, the light emitting diodes 22 and one or more sensors 24 can be included in a wearable structure, for example, similarly to a watch. This wearable structure can also include the processor 4, or the wearable structure can be configured to transmit data to the processor 4 that is external to the wearable structure.

Regardless of probe 2 structure, during operation of the disclosed device in determining oxy-hemoglobin level, deoxy-hemoglobin level, and/or a ratio of oxy-hemoglobin to deoxy-hemoglobin, the light of the plurality of light emitting diodes 22 is directed towards a portion of the mammal's skin.

If that mammal has a relatively low hemoglobin level, a greater proportion of the light emitted from the light emitting diodes 22 is reflected back and received by the one or more sensors 24. For example, more light emitted from the light emitting diodes 22 passes through the epidermis and reflects off of both the upper vascular plexus and the lower vascular plexus, and then back again through the epidermis. Thus, in this example, less light is absorbed in the dermal papillae between the lower vascular plexus and the upper vascular plexus, and between the upper vascular plexus and the epidermis.

In contrast, if that mammal has a relatively high hemoglobin level, a lesser proportion of the light emitted from the light emitting diodes 22 is reflected back and received by the one or more sensors 24. For example, less light emitted from the light emitting diodes 22 passes through the epidermis and reflects off of both the upper vascular plexus or lower vascular plexus, and then back again through the epidermis. Thus, in this example, more light is absorbed in the dermal papillae between the lower vascular plexus and the upper vascular plexus, and between the upper vascular plexus and the epidermis.

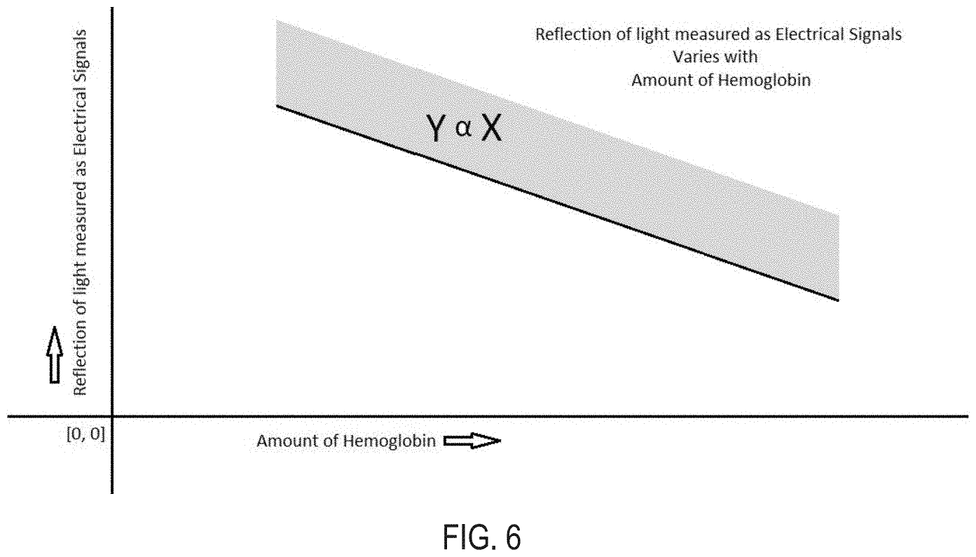

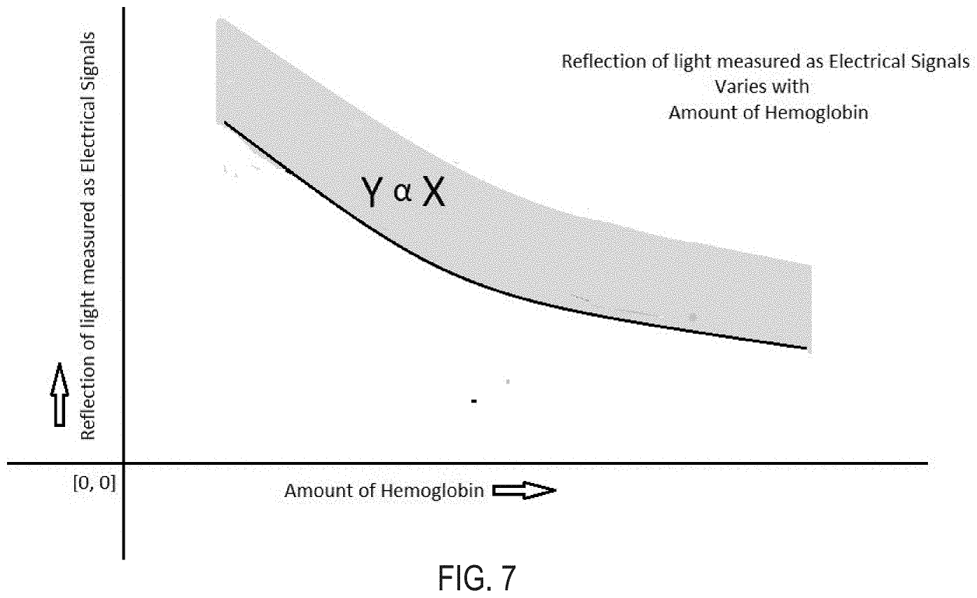

The relationship between the reflection of light and hemoglobin levels is discussed below in reference to FIGS. 6 and 7.

The relationship between the amount of reflection of light measured as an electrical signal (current) (on the Y-axis) and amount of hemoglobin in the blood (on the X-axis) is shown in FIGS. 6 and 7. These graphs are based on several readings from mammals, humans specifically, with different skin color. Mammals with a lighter skin have less melanin in their skin, as compared to mammals with darker skin. In FIG. 6 skin color is kept constant, so that the relationship between electrical signal and the hemoglobin concentration is linear.

Specifically in FIG. 6, the graph illustrates the relationship between the amount of reflection of light measured as an electrical signal (current) (on the Y-axis) by the one or more sensors 24 and amount of hemoglobin in the blood (on the X-axis) of the mammal. The relationship is substantially linear as indicated by the straight line, which applies under the assumption that melanin concentration in the skin of the mammal (or the skin of a plurality of mammals) is substantially constant. The grey shaded area above the substantially straight line represents the symmetrical vertical shift in this linear relationship depending on the skin melanin concentration.

In partial contrast to FIG. 6, as seen in FIG. 7 a graph illustrates the relationship between the amount of reflection of light measured as an electrical signal (current) (on the Y-axis) by the one or more sensors 24 and amount of hemoglobin in the blood (on the X-axis) of the mammal. The line in FIG. 7 is substantially logarithmic due the varying melanin levels of the skin of the humans measured to arrive at the data. The grey shaded area above represents the vertical shift in this relationship depending on the variability in the skin melanin concentration of the mammal under measurement.

The difference between FIG. 6 and FIG. 7 demonstrates the impact melanin content of the mammal's skin can have on the accuracy of a hemoglobin reading.

As a further demonstration of the effect melanin has on hemoglobin determinations, data is presented and discussed with reference to FIGS. 7-10.

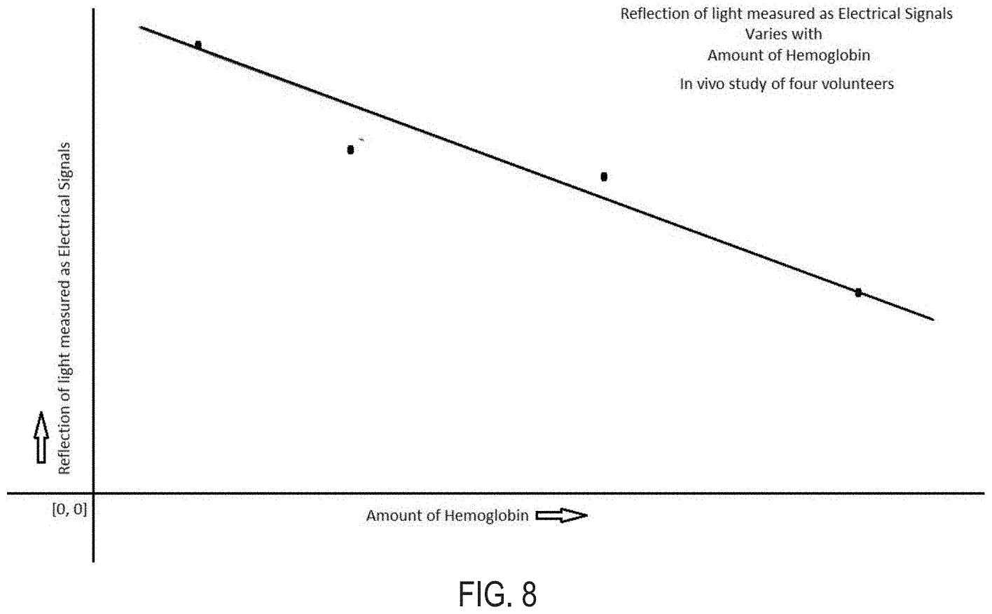

FIG. 8 is the graphical representation of data from four different individuals, with similar melanin content (as measured on the Felix Von Luschan (VLS) skin color chart), using the probe 2 on the same site in each individual (back of the wrist). The VLS scale provides a correlation of the color grade of a human's skin (from 1-36) to the estimated melanin content of that human's skin. Thus, the VLS scale can be relied upon to provide a melanin content substantially accurately for a human based on their assignment to one of 36 skin colors.

Referring again to FIG. 8, the amount of reflection of light measured as an electrical signal (current) is plotted on the Y-axis, measured by the one or more sensors 24, and amount of hemoglobin (measured using the conventional blood draw method) in the blood of the individual is plotted on the X-axis. The relationship is substantially linear as shown with the substantially straight line. As a comparison, the blood hemoglobin of the four different individuals was also measured using the standard cyanmethemoglobin laboratory technique, as shown by the four data points in FIG. 8. As can be seen, there is a substantially accurate correlation between measured light values translated to hemoglobin content as compared to blood tested hemoglobin levels.

As one way to correct for the influence varying melanin levels has on optical hemoglobin determinations, the probe 2 could be made so that the nozzle 23 has a relatively small opening size. Under this embodiment, the melanin concentration of the subject's skin is finite and defined, and if the same amount of light is passed from the plurality of LEDs 22 to a smaller skin surface are of the mammal, the emitted light will encounter a relatively smaller amount of melanin. Since the concentration of melanin is typically in the microgram range, and the concentration of hemoglobin is typically in the gram range, the impact of varying melanin concentrations on optical hemoglobin determinations can be minimized.

As another way to correct for the influence varying melanin levels has on optical hemoglobin determinations, melanin levels can be considered, as discussed below.

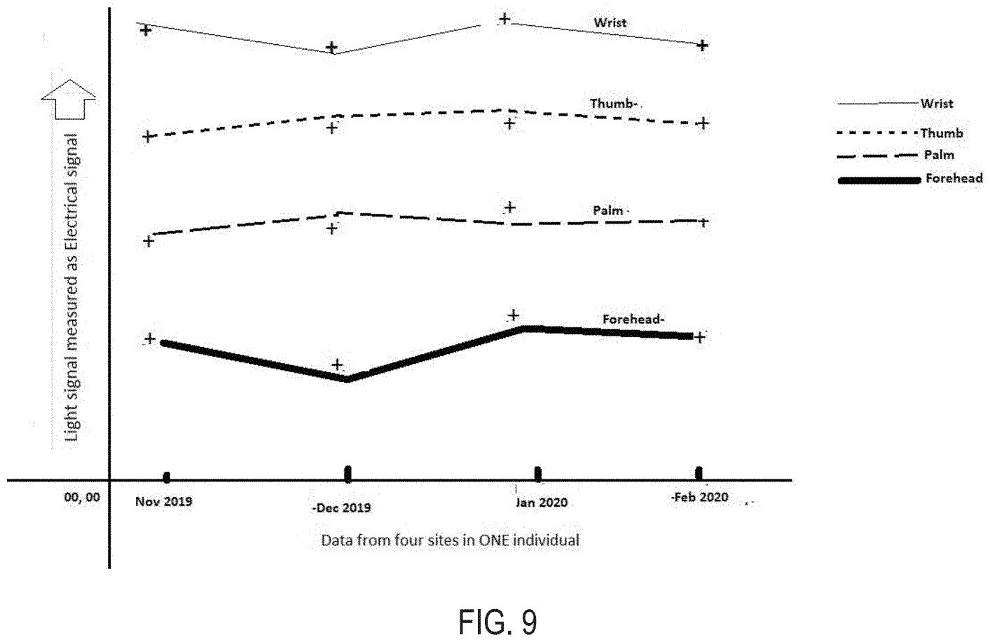

In another example, FIG. 9 is the graphical representation of the temporal and spatial data from one individual, using the probe 2 on four different sites at four different time points, each of which were about four weeks apart. The data points, shown as crosses, are the result of a blood draw and laboratory determination of hemoglobin (substantially constant at 14.4 g/dL).

The amount of reflection of light measured as an electrical signal (current) by the one or more sensors 24 is plotted on the Y-axis and the corresponding time point is plotted on the X-axis. As seen from the graph, the readings are substantially reproducible temporally across 4 different timepoints for the same site. However, there is significant difference across readings obtained from different sites. For example, the amount of reflected light from the wrist is higher and it progressively decreases from wrist-thumb-palm-forehead. Therefore, each probe 2 can be designed for a specific reading location, or the probe 2 and processor 4 can be adjusted to account for the different reading location.

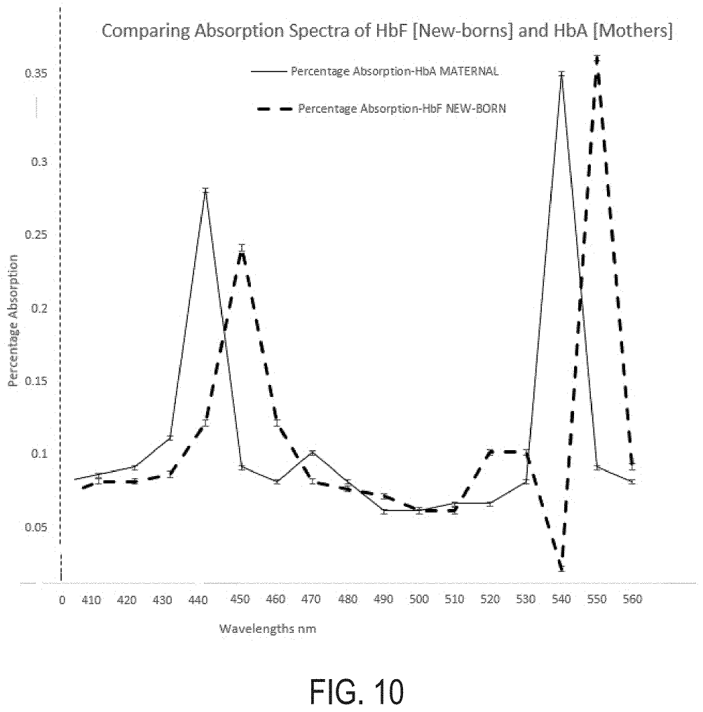

FIG. 10 is the graphical representation of data from 25 mother-infant pairs, to demonstrate similar spectroscopic properties of fetal hemoglobin (Hb-F) and adult hemoglobin (Hb-A), using the disclosed device. Hb-F is the predominant form of the hemoglobin in newborns and is gradually replaced by Hb-A by 8-9 months of age. The percentage of absorbed light is plotted on the Y-axis and the corresponding wavelength (in nanometers) is plotted on the X-axis. Hb-F and Hb-A have remarkably similar spectroscopic properties with the absorption peaks at 450-460 nm and 540-550 nm. Due to similar spectroscopic properties of blood hemoglobin-A and hemoglobin-F, the disclosed device can be used to measure hemoglobin concentration in adults and infants that are younger than 9 months.

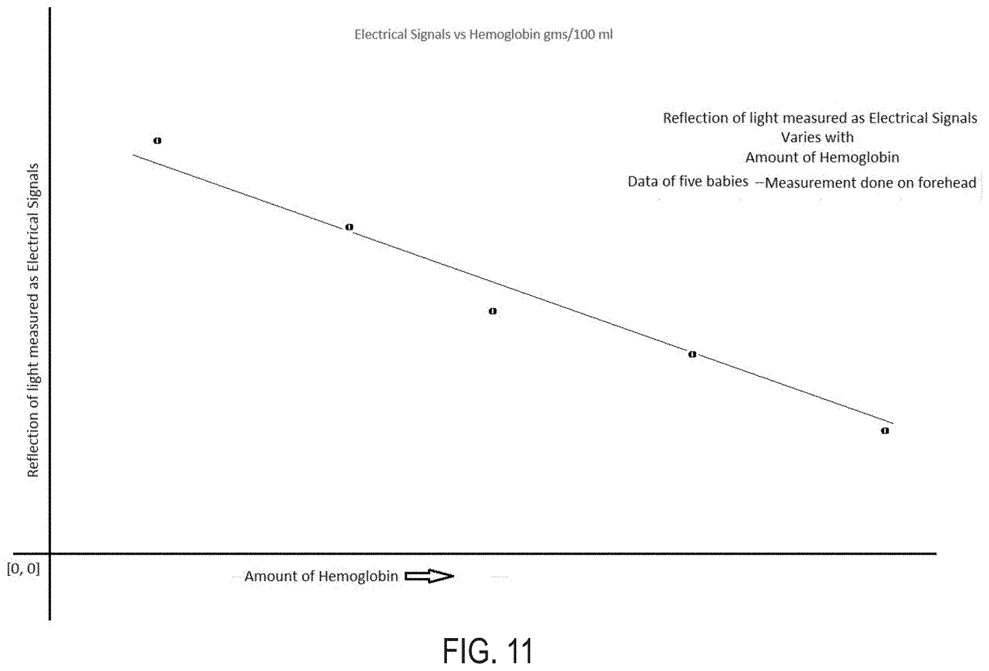

As a further demonstration of this, FIG. 11 is the graphical representation of data from 5 infants, with similar melanin content (as measured on the VLS scale), using the probe 2 on the same site for each infant (forehead). The amount of reflection of light measured as an electrical signal (current) by the one or more sensors 24 is plotted on the Y-axis and amount of hemoglobin in the blood is plotted on the X-axis. The five data points indicated by small circles are the laboratory results of a typical blood draw hemoglobin test. The relationship is substantially linear as shown by the substantially straight line, with the laboratory data being near the linear measurement results of the disclosed device.

Further, Table 2 below includes data from four human subjects, with light brown skin color (VLS scale 24-25). Corresponding R values are calculated using the formula, R=E*H. This E was obtained using the disclosed device, and the blood hemoglobin concentration (H) was obtained using the conventional blood draw method. In Table 2, as well as the rest of the disclosure, the “Ratio” of E1 or E2 is the ratio of light emitted by the plurality of LEDs 22 at that wavelength as compared to the amount detected by the at least one sensor 24 at that wavelength.

TABLE 2

| | | | | Hb (H) | |
|---|---|---|---|---|---|
| | | | Total | measured by | |
| | Ratio | Ratio | Ratio | blood draw | R |
| Subject | E1 | E2 | E = E1 + | (grams/ | value = |
| no. | (525 nm) | (545 nm) | E2 | 100 ml) | E*H |
| 1 | 0.692 | 0.671 | 1.363 | 13.71 | 18.69 |
| 2 | 0.736 | 0.749 | 1.485 | 12.29 | 18.25 |
| 3 | 0.633 | 0.616 | 1.249 | 14.56 | 18.19 |
| 4 | 0.650 | 0.626 | 1.276 | 14.32 | 18.27 |

E1 = Ratio of reflected light using the 525 nm LED of the disclosed device
E2 = Ratio of reflected light using the 545 nm LED of the disclosed device

In Table 2 above and throughout the disclosure, the constant ‘R’ is related to the hemoglobin but also to the skin color or skin pigment melanin. The constant R would be a function of the amount of skin melanin and/or hemoglobin in the given subset of race/ethnicity. Therefore, this factor “R” would be different across different races/ethnicities.

R can be further depicted as, R=kM Where ‘M’ represents variable concentration of Melanin in the skin and ‘k’ is the constant factor related to the hemoglobin.

Due to two variables E (either 1 or 2) and M, the nature of the mathematical relationship between E and H could be substantially linear or substantially logarithmic, and the relationship could be depicted using a substantially logarithmic scale (FIG. 7 noted above) as: R=log (E*H)

The value of R (since R=kM) is dependent on the amount of melanin “M” in the skin. If the value of M is kept substantially constant, the value of R would be substantially constant. In other words, the value of R will be substantially constant for the subjects with the same skin melanin concentration (M). In such case, the relationship would tend to be substantially linear, and can be expressed (FIG. 6) as: R=E*H

Because the disclosed device measures E at various wavelengths, the amount of hemoglobin H (in grams/100 ml) in the blood can be calculated by the processor 4, with the formula: H (in grams/100 ml)=R/E

The value of R can be obtained through a melanin concentration determination. As noted above, the value of R is variable depending on the skin melanin concentration and would be constant for a specific melanin concentration. Subjects with lighter skin color (with less melanin, such as Caucasian human subjects) have higher R values, compared to the subjects with the darker skin color (with more melanin, such as African American human subjects), with R being substantially constant for subjects with the same or similar melanin concentrations.

The mean and the standard deviation of the R values obtained in Table 2 were calculated. The mean calculated R value, with the standard deviation (S.D), in this case is (mean=18.35, S.D=0.23). Using a 95% confidence interval (CI) for the data of Table 2 case would be: Mean+/−2 S.D for 95% CI is (17.89-18.81). Therefore, the R value for human subjects with the light skin tone (VLS 24-25) is expected to be around 18.35 and would fall between the intervals of 17.89 to 18.81 for at least 95% of the subjects measured.

Similarly, to Table 2 above, Table 3 below is a representation of data from three human subjects, with dark brown skin color (VLS scale 30-31). Corresponding R values were calculated using the formula, R=E*H. Although only one time point is listed in Table 3 for each of the subjects, in other examples, multiple measurements can be undertaken for each subject. These multiple measurements can span the time ranges noted above, such as continuous or near continuous measurement, up to a measurement every several minutes or more.

TABLE 3

| | | | | Hb (H) | |
|---|---|---|---|---|---|
| | | | Total | measured by | |
| | Ratio | Ratio | Ratio | blood draw. | R |
| Subject | E1 | E2 | E = E1 + | (grams/ | value = |
| no | (525 nm) | (545 nm) | E2 | 100 ml) | E*H |
| 1 | 0.360 | 0.368 | 0.728 | 15.71 | 11.43 |
| 2 | 0.396 | 0.395 | 0.791 | 14.60 | 11.55 |
| 3 | 0.382 | 0.367 | 0.749 | 16.30 | 12.21 |

E1 = Ratio of reflected light using the 525 nm LED of the disclosed device
E2 = Ratio of reflected light using the 545 nm LED of the disclosed device

Similarly, to the procedure in Table 2, discussed above, for the data of Table 3, the calculated mean and S.D was (mean=11.73, S.D=0.42). Thus, using a 95% confidence interval (CI) for Table 3, Mean+/−2 S.D for 95% CI is (10.89-12.57). Therefore, the R value for subjects with the dark skin tone (VLS scale 30-31) is expected to be around 11.73 and would fall between the intervals of 10.89 to 12.57 for at least 95% of the subjects measured.

Table 4 is a representation of data from two human subjects. Subject X with light brown skin color (VLS grade 24) and subject Y with dark brown skin color (VLS grade 30).

TABLE 4

| | | | | | | Total | | |
|---|---|---|---|---|---|---|---|---|
| | | | | | R value for | hemoglobin (H, | | |
| | | | | | each skin color | (grams/100 ml) | Hemoglobin | |
| | | Ratio | Ratio | Total | obtained from | calculated by | measured | Difference |
| | | E1 | E2 | Ratio | previous | the device using | using blood | (grams/ |
| | Skin | (525 | (550 | E = | experiments | H = R/E with | draw. | 100 ml) |
| Subject | color | nm) | nm) | E1 + E2 | with (95% C.I.) | (95% C.I.) | (grams/100 ml) | %) |
| X | Light | 0.668 | 0.672 | 1.340 | 18.35 | 13.69 | 13.90 | (−0.21, |
| | | | | | (17.89-18.81) | (13.35-14.0) | | 1.5) |
| Y | Dark | 0.411 | 0.396 | 0.807 | 11.73 | 14.53 | 14.80 | (−0.27, |
| | | | | | (10.89-12.57) | (13.49-15.58) | | 1.82) |

E1 = Ratio of reflected light using the 525 nm LED of the disclosed device.
E2 = Ratio of reflected light using the 545 nm LED of the disclosed device.
95% C.I. = 95% confidence interval obtained by Mean ± 2 (Standard deviation).

In Table 4, for each subject, values of E were obtained using the disclosed device (column 4). Next, depending upon the skin color, corresponding mean R values (with 95% C.I.) for each skin color were chosen as shown in column 5 (based on Tables 2 and 3 above). Hemoglobin values were calculated using the formula, H=R/E, and are shown in column 6 along with the limits of agreement. These values were compared to those obtained by the conventional blood draw method (column 7).

As shown in column 8, the differences between the hemoglobin values obtained using the disclosed device and conventional blood draw are remarkably small and fall well within the limits of agreement. Specifically, for both subject X and subject Y, the disclosed device was able to accurately estimate the blood hemoglobin value. The calculated values 13.69 (grams/100 ml) (for Subject X) and 14.53 (grams/100 ml) (for Subject Y) are close to the values measured by the conventional blood draw method (13.90 (grams/100 ml) and 14.8 (grams/100 ml) respectively). In both examples, the difference between the device calculated hemoglobin value and the traditional (blood) draw measured value was about 0.2-0.3 (grams/100 ml). This difference is small and is significantly less than the currently available, approved by the Food and Drug Administration (FDA), non-invasive devices.

As a further example, multiple readings were obtained from a number of subjects with different skin colors using the disclosed device on the back of each of their wrists. The R value and mean were calculated for each individual relied on for data in Tables 2 and 3.

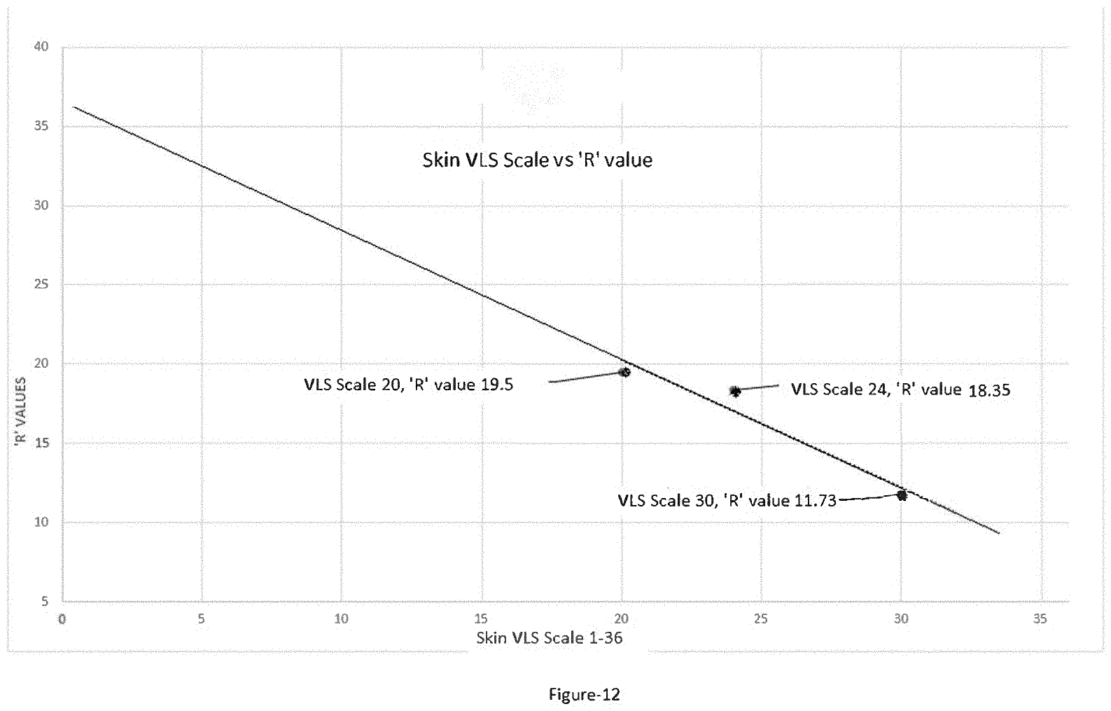

The average R was then plotted on the Y-axis and corresponding skin color (categorical VLS scale from 1-36) on the X-axis. These results are illustrated in FIG. 12.

For subjects with light skin color (VLS 20), the mean R value was 19.5. For subjects with slightly darker skin (VLS 24), the mean R value was 18.35. The R value further decreases (mean value 11.73) for subjects with very dark skin (VLS 30). This data demonstrates a relatively consistent, substantially linear inverse relationship between the VLS scales and the R value. These findings support that the R value is significantly lower (around 5-10) in subjects with higher VLS scales as compared (around 35-40) to subjects with lower VLS scales.

Another embodiment of the present disclosure is directed to devices and methods for measuring creatinine in a mammal's blood. Determining creatinine level in blood can be an indicator of how well the mammal's kidneys are performing. Creatinine is, generally, a chemical compound created during energy-producing processes in the muscles. Kidneys that are performing healthily typically filter a portion, majority, nearly all or all of creatinine out of the mammal's blood. In contrast, a higher detectable level of creatinine indicates kidneys that are performing less healthily.

The amount of creatinine in the blood is relatively stable in healthy mammals. Serum creatinine is typically reported as milligrams of creatinine to a deciliter of blood (mg/dL) or micromoles of creatinine to a liter of blood (micromoles/L). The typical range for serum creatinine is 0.6-1.2 mg/dL.

Determination of creatinine levels in blood is important for several conditions, including but not limited to: diagnosing kidney disease; screening for kidney disease in conditions such as diabetes, high blood pressure or other conditions that increase the risk of kidney disease; monitoring kidney disease treatment or progression; monitoring for side effects of drugs that may include kidney damage or altered kidney function; monitoring the function of a transplanted kidney; and monitoring kidney function in patients requiring hemodialysis for acute and/or chronic kidney failure, for example.

Similar to the reflectance principles discussed herein, a creatinine probe device can be utilized to measure reflected light to determine creatinine levels in blood.

Creatinine has several peaks within the light absorption spectrum, however, the following peaks were selected: about 785 nm to about 845 nm, about 790 nm to about 840 nm, about 795 nm to about 835 nm, about 800 nm to about 830 nm, about 805 nm to about 825 nm, about 810 nm to about 820 nm or about 815 nm. Thus, one or more light emitting diodes can be used to emit light at one or more of these wavelengths, which provides a benefit because at these wavelengths there is little or no light absorption and interference due to chromophores within the mammal's skin, such as melanin and/or hemoglobin.

In this embodiment, directed to creatinine detection, the probe 2 of FIG. 2 can also be used. In FIG. 2, within the probe housing 20 are a plurality of light emitting diodes 22. The wavelengths of each of the plurality of these light emitting diodes 22 can be the same or different, can be fixed or variable, and can be any suitable wavelength, in any suitable range, such as about 780 nm to about 840 nm. Specific examples of such wavelengths include, but are not limited to about 810 nm, about, 815 nm, and about 830 nm. However, in other embodiments, the wavelengths can differ from any of the above values by about 0.001%, about 0.01%, about 0.1%, about 0.5%, about 1%, about 2%, about 3%, about 4%, about 5%, about 7%, about 9%, about 10%, about 13%, about 15%, about 20% or more.

Using the probe 2, if the mammal has a relatively high creatinine level, a lesser proportion of the light emitted from the light emitting diodes 22 is reflected back and received by the one or more sensors 24. Example 3 below discusses use of probe 2 and creatinine detection in more detail.

Another embodiment of the present disclosure is directed to devices and methods for measuring glucose in a mammal's blood. Determining glucose level in blood can be an indicator of how well the mammal's body is managing glucose and insulin levels during any illness and/or activity.

The American Diabetes Association recommend target levels of 70-130 mg/dL before eating for a person with diabetes. Within 2 hours of eating a meal, blood glucose levels should be less than 180 mg/dL.

Health authorities consider a normal fasting blood sugar level between 60-99 milligrams per deciliter (mg/dL). In people with diabetes mellitus, the levels are typically higher. Frequent monitoring of blood glucose is used for clinical management of diabetes mellitus. Similarly frequent monitoring of blood glucose is used in critical care illness, liver failure, drug overdose, and for athletic performance.

Similar to the reflectance principles discussed herein, a glucose probe device can be utilized to measure reflected light to determine glucose levels in blood.

Glucose has several peaks within the light absorption spectrum, however, the following peaks were selected; about 900 nm to about 1,200 nm, or about 910 nm to about 1,190 nm, or about 920 nm to about 1,180 nm, or about 930 nm to about 1,170 nm, or about 940 nm to about 1,160 nm, or about 950 nm to about 1,150 nm, or about 960 nm to about 1,140 nm, or about 970 nm to about 1,130 nm, or about 980 nm to about 1,120 nm, or about 990 nm to about 1,110 nm, or about 1,000 nm to about 1,100 nm, or about 1,050 nm. Thus, one or more light emitting diodes can be used to emit light at one or more of these wavelengths, which provides a benefit because at these wavelengths there is little or no light absorption and interference due to chromophores within the mammal's skin, such as melanin and/or hemoglobin.

In this embodiment, directed to glucose detection, the probe 2 of FIG. 2 can also be used. In FIG. 2, within the probe housing 20 are a plurality of light emitting diodes 22. The wavelengths of each of the plurality of these light emitting diodes 22 can be the same or different, can be fixed or variable, and can be any suitable wavelength, in any suitable range, such as the peak ranges for glucose noted above. However, in other embodiments, the wavelengths can differ from any of the above values by about 0.001%, about 0.01%, about 0.1%, about 0.5%, about 1%, about 2%, about 3%, about 4%, about 5%, about 7%, about 9%, about 10%, about 13%, about 15%, about 20% or more.

Example 4 below discusses use of probe 2 and glucose detection in more detail.

Another embodiment of the present disclosure is directed to devices and methods for measuring lactate in a mammal's blood. Lactate has several peaks within the light absorption spectrum, however, the following first peaks were selected: about 1490 nm to about 1610 nm, or about 1500 nm to about 1600 nm, or about 1510 nm to about 1590 nm, or about 1520 nm to about 1580 nm, or about 1530 nm to about 1570 nm, or about 1540 nm to about 1560 nm, or about 1550 nm.

The following second peaks were also selected: about 1650 nm to about 1810 nm, or about 1670 nm to about 1790 nm, or about 1690 nm to about 1770 nm, or about 1710 nm to about 1750 nm, or about 1720 nm to about 1740 nm, or about 1730 nm.

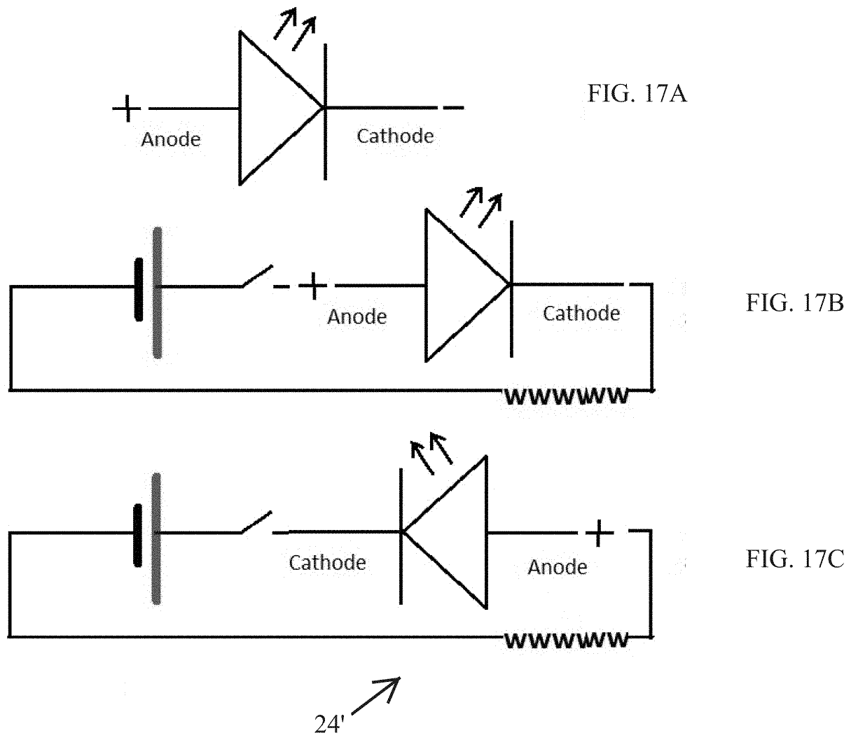

Measurement of wavelengths in these ranges is impractical and/or impossible for known optical sensors. Specifically, exact quantification of electromagnetic radiations [EMR] in these ranges is hindered by non-availability of specific optical sensors. Therefore, the one or more sensors 24 of FIG. 2 can be replaced with one or more sensors 24′ as discussed with reference to FIGS. 17A-17C.

Light Emitting Diodes (LEDs) are small components which emit light of specific wavelengths, an example of which is shown schematically in FIG. 17A. This is specific for each LED due to its unique structure. The forward bias mode, shown in FIG. 17B, refers to the typical use of LED with the anode provided with a positive electrical charge and cathode connected to a negative electrical charge. This ensures flow of electric current in the LED, creating an electrochemical gradient, specific for each LED due to its unique structure. This leads to LED emitting the desired wavelength of the light.

Optical sensors, such as the sensors 24, are the components which sense optical (light) signals and convert them into the electrical signals (electric current). This electric current is amplified and then measured to inform the intensity of received light by the optical sensor.

Typically, LEDs convert current into light, whereas sensors convert light into the current. However, if LEDs are used in the reverse bias mode (anode connected to the negative electrical charge and cathode connected to the positive electrical charge), an LED could act as optical sensor, such as sensor 24′ shown in FIG. 17C, for the specific range of wavelength they are configured to emit.

For example, an LED of FIG. 17B designed to emit 600 nm (red light) wavelength, by using this LED in the reverse bias mode as sensor 24′ of FIG. 17C, the sensor 24′ can sense the light wavelengths in the range of about 550 nm to about 600 nm. Therefore, to detect a specific light wavelength of 1550 nm with a sensor 24′ of FIG. 17C, an LED of FIG. 17B, of about 1600 nm wavelength can be used. The optical signal detected by sensor 24′ can then subsequently be converted to electrical signals (current).

In this embodiment, directed to lactate detection, the probe 2 of FIG. 2 can be used. In FIG. 2, within the probe housing 20 are a plurality of light emitting diodes 22, and one or more sensors 24 or 24′. The wavelengths of each of the plurality of these light emitting diodes 22 can be the same or different, can be fixed or variable, and can be any suitable wavelength, in any suitable range, such as those in or near the peak ranges noted above. However, in other embodiments, the wavelengths can differ from any of the above values by about 0.001%, about 0.01%, about 0.1%, about 0.5%, about 1%, about 2%, about 3%, about 4%, about 5%, about 7%, about 9%, about 10%, about 13%, about 15%, about 20% or more.

Example 5 below discusses use of probe 2 and lactate detection in more detail.

### EXAMPLE 1

In this example, a patient enters a clinical setting for a hemoglobin level determination. The operator then places the probe 2 on a portion of the patient, for example the patient's wrist.

The one or more sensors 24 detect reflected light, in this example at 525 nm and 545 nm, such that the processor 4 can determine the ratio of both of E1 and E2. The processor 4 makes this determination by determining the ratio of returned light detected by the one or more sensors in comparison to the emitted light from the plurality of LEDs 22, at each wavelength.

The processor 4 then outputs two ratios (E1) and (E2). The processor 4 can then add those values (E1)+(E2) (or a user can manually add those ratios) to determine a total E value. Next, the VLS scale value is determined in one of two ways.

The first way is for a user to estimate the value by a visual inspection and assignment of the patient to a score of 1-36 on the VLS scale. Under this first option, the operator can then manually select the corresponding R value for the selected score (present on a provided chart that includes all VLS scale scores and their corresponding R values). The operator can then manually divide the R value by E.

The second way is for the probe 2 to include an optical sensor (one or more sensors 24, or an additional sensor) that can receive a signal, and the processor 4 can, based on the signal, automatically assign the patient to a VLS scale value (with its corresponding R value), with those VLS scale values and R values being stored in the electronic storage device 10. The processor 4 can then divide the R value by the obtained E value to determine the total hemoglobin value.

### EXAMPLE 2

In addition to the total hemoglobin as described above in Example 1, the disclosed device can also measure the ratio of oxy-hemoglobin [O-Hb] to deoxy-hemoglobin [d-Hb], as well as change in the ratio, as discussed in this example.

In this example, a patient enters a clinical setting. The operator then places the probe 2 on a portion of the patient, for example the patient's wrists. The total hemoglobin value is then obtained as discussed in Example 1.

Further, since E1 substantially corresponds to the data collected at the 525 nm wavelength, it is indicative of oxy-hemoglobin concentration, while E2, substantially corresponding to data collected at the 550 nm level (in this example, however, about 545 nm level can also be used for data collection), is indicative of deoxy-hemoglobin concentration. The ratio of E1 to E2 can be determined once, or sequentially to monitor the patient's condition.

In this example, blood hemoglobin was measured with the disclosed device for a patient presenting an acute asthma attack. Asthma causes airways to become inflamed and constrict, leading to low oxygen concentration in the blood. The patient had a VLS skin scale of 20, with the corresponding R value of 19.5 based on a value demonstrated by FIG. 12.

At first, the patient had symptoms of breathlessness and cough. The obtained readings are depicted immediately after presentation, at time 2 minutes (Table 5), with the disclosed device. Patient was then observed and treated with medications to relieve cough and breathlessness. However, patient's clinical condition deteriorated. Another set of readings is obtained at 21 minutes with the disclosed device. Patient was subsequently treated with a supplemental oxygen face mask. Patient's condition improved, and a repeat set of observations were obtained after about 20 minutes of oxygen therapy (at 40 minutes), with the disclosed device.

TABLE 5

| | | | | | R | Calculated Hb | Measured Hb | Oxygen |
|---|---|---|---|---|---|---|---|---|
| | Ratio | Ratio | | | value | using the | using the | saturation using |
| | E1 | E2 | | Total | for | device. | blood draw | arterial blood |
| Time | (525 | (550 | Ratio | ratio | skin | H = R/E | method. | gas (normal |
| (mins) | nm) | nm) | E1/E2 | (E1 + E2) | VLS 20 | (grams/100 ml) | (grams/100 ml) | 97-100%) |
| 2 min | 0.631 | 0.608 | 1.038 | 1.239 | 19.50 | 15.74 | 15.30 | 90% |
| (room air) | | | | | | | | |
| 21 min | 0.652 | 0.588 | 1.109 | 1.240 | 19.50 | 15.73 | 15.40 | 86% |
| (room air) | | | | | | | | |
| 40 min | 0.615 | 0.622 | 0.988 | 1.237 | 19.50 | 15.76 | 15.30 | 96% |
| (Oxygen face | | | | | | | | |
| mask) | | | | | | | | |

E1 = Ratio of reflected light using the 525 nm LED of the disclosed device
E2 = Ratio of reflected light using the 545 nm LED of the disclosed device

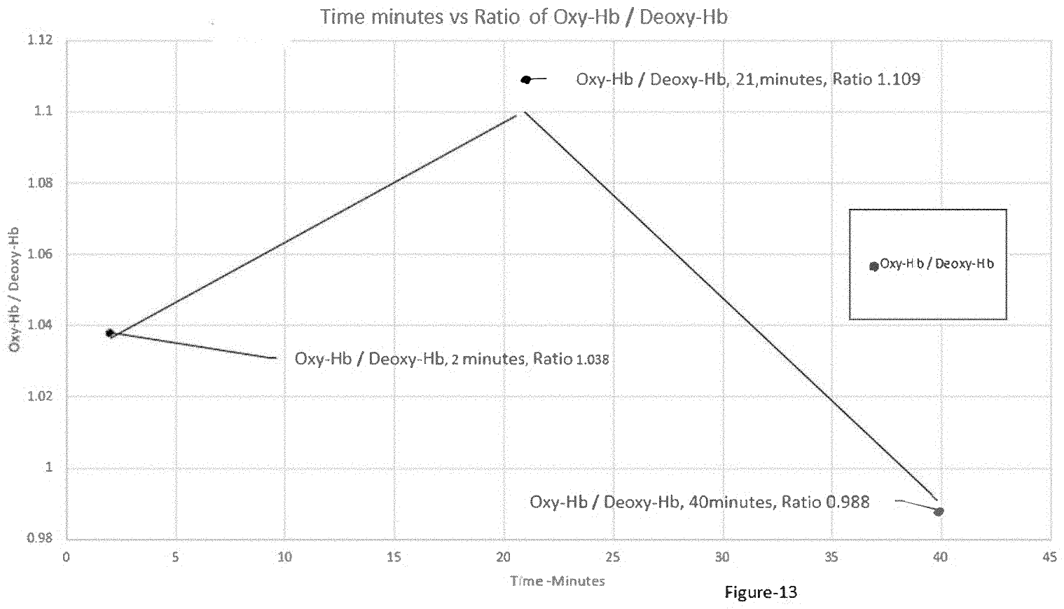

The E1 divided by E2 ratio was obtained at each time point by the processor 4. As seen from Table 5, the ratio increased from 1.038 to 1.109 as patient's clinical condition worsened. After treatment with the supplemental oxygen, patient's condition improved, and the E1/E2 ratio decreased to 0.988. These results are shown in the FIG. 13.

Specifically, in FIG. 13, the ratio of E1/E2 measured using the disclosed device, is plotted on the Y axis, and the corresponding time point (in minutes) is plotted on the X axis. At presentation, the patient had hypoxia (low oxygen content), with E1/E2 ratio 1.038. After 20 minutes, patient's condition and hypoxia worsened, with corresponding increase the ratio to 1.109. Patient was subsequently treated using supplemental oxygen. Patient's hypoxia improved, correlated with the ratio of 0.988.

As seen in FIG. 13, as the ratio of E1/E2 increases, the blood oxygen saturation (an indicator of oxyhemoglobin) decreases. Since E and H have an inverse relationship as previously described (FIG. 6), the ratio E1/E2 is expected to have an inverse relationship with the relative oxy-hemoglobin (O-Hb) concentration. Oxy-hemoglobin is the form of hemoglobin attached to the oxygen molecules and is responsible for the oxygen delivery to the tissue.

Therefore, ratio E1/E2 provides a tool to monitor patient's clinical condition without requiring the need for the invasive arterial blood gas sampling. The ratio can be calculated at the bedside by a user, or the processor 4 could determine this ratio. Currently available non-invasive method (pulse-oximetry) to measure blood oxygen saturation, has limitations in dark-skinned subjects, and during low perfusion states, such as shock. The disclosed device does not have these limitations.

Since the relative ratios of oxy and deoxy hemoglobin are unique for each individual, the absolute value of ratio E1/E2 would vary between individuals. However, the ratio E1/E2 would be specific for the given subject and could be used through serial measurements as a non-invasive means of oxyhemoglobin monitoring. This can significantly decrease the need for the invasive blood collection.

### EXAMPLE 3

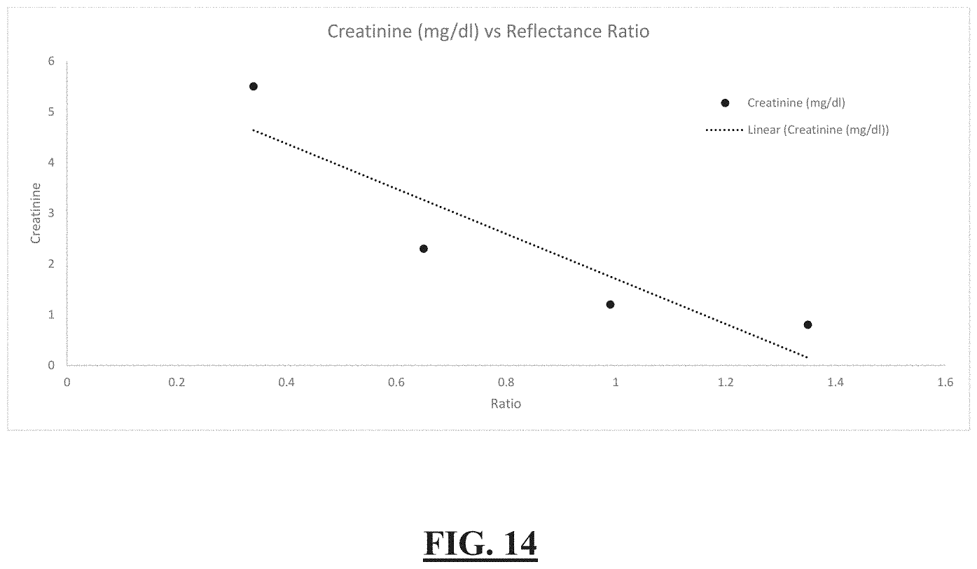

Using a device disclosed above, the reflectance ratio of 4 human subjects was estimated with a detection wavelength of about 815 nm. In other embodiments, the detection wavelength can be about 805 nm to about 825 nm. The creatinine values were also measured nearly simultaneously using a known blood draw method. The results are shown in FIG. 14.

Based on these observations, a proposed equation for calculation of blood creatinine using the disclosed device can be depicted as the following creatinine value formula:

Creatinine value=6.15-4.45 (Ratio)

To test the accuracy of the equation, blood creatinine values of a subject undergoing hemodialysis were estimated. The creatinine values were estimated using the disclosed device, and also measured by a nearly simultaneous, known blood draw method, both before and after the dialysis. Blood creatinine values typically decline after a dialysis session. The results are shown in Table 6 below:

TABLE 6

| | Ratio | Creatinine | Creatinine |
|---|---|---|---|
| | using | estimated using | measured by the |
| | disclosed | the disclosed | blood draw |
| Time | device | device (mg/dl) | method (mg/dl) |
| Before dialysis | 0.48 | 4.0 | 4.3 |
| After dialysis | 0.75 | 2.8 | 2.5 |

As demonstrated in Table 6, the disclosed device can accurately measure the creatinine values both before and after the dialysis as compared to the invasive, known, blood draw method. Significantly, the disclosed device is sensitive to detect minor changes and can be used for point of care estimation, determination and monitoring of blood creatinine.

### EXAMPLE 4

### Experiment-1

In the first experiment, a diabetic subject participated in this observational experiment. Four readings were obtained by the disclosed device, and corresponding glucose values were measured. The readings were done just before lunch and at intervals of 30 minutes for 2 hours. Observations are tabulated in Table 7. In Table 7, the wavelength that was emitted from the one or more light emitting diodes (22) was 1050 nm using the disclosed device of FIG. 2, and the wavelength that was used to detect the reflected light from the person's skin, with the one or more sensors (24) was 1050 nm using the disclosed device of FIG. 2. Table 7 is below:

TABLE 7

| | Reflection | Blood sugar | Lab test |
|---|---|---|---|
| Timeline | Ratio (E) | (mg/dl) | mg/dl |
| 0 Just before lunch | 0.74 | 184 | 179 |
| 30 min after lunch | 0.68 | 210 | 223 |
| 60 min after lunch | 0.67 | 224 | 204 |
| 120 min after lunch | 0.79 | 177 | 183 |

### Experiment-2

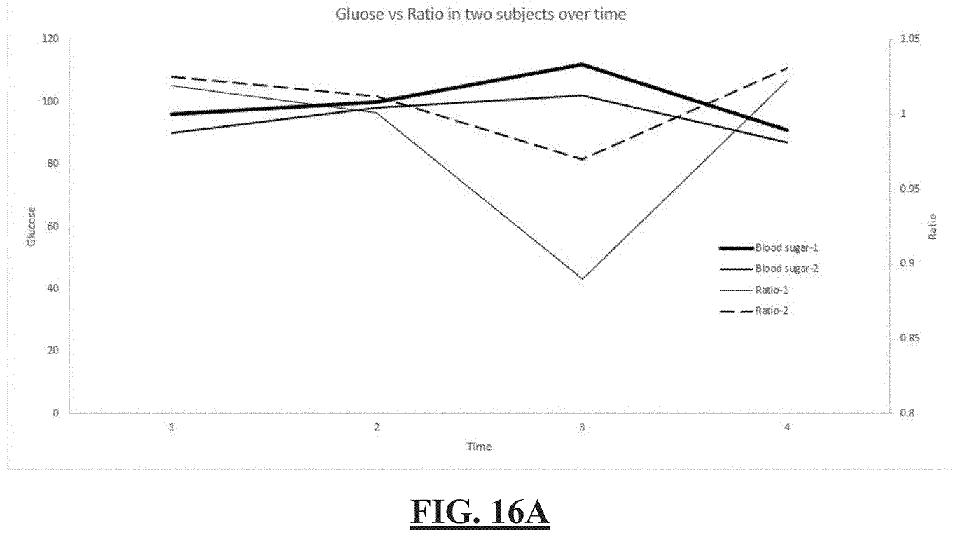

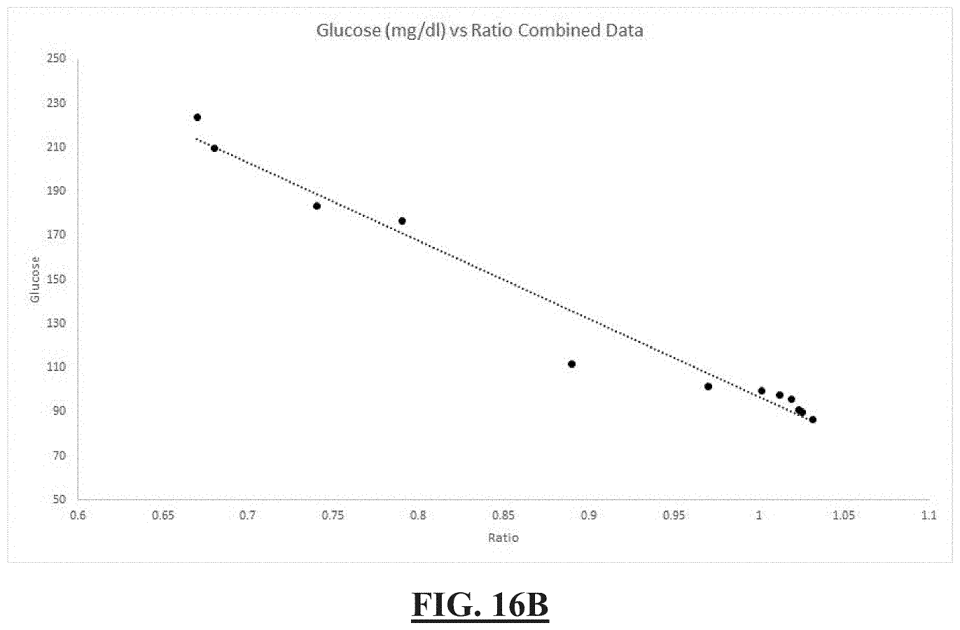

In the second experiment, two nondiabetic subjects participated in this observational experiment. Four readings were obtained by the disclosed device, and corresponding glucose values were measured. The readings were done just before lunch and at intervals of 30 minutes for 2 hours. Observations are tabulated below in Table 8 and illustrated in FIG. 16A:

TABLE 8

| | | Blood | | Blood |
|---|---|---|---|---|
| | E ratio | glucose | E ratio | sugar |
| Timeline | (Subject-1) | (subject 1) | (subject-2) | (subject-2) |
| 0 Just before lunch | 1.019 | 96 | 1.025 | 90 |
| 30 min after lunch | 1.001 | 100 | 1.012 | 98 |
| 60 min after lunch | 0.89 | 112 | 0.97 | 102 |
| 120 min after lunch | 1.023 | 91 | 1.031 | 87 |

Both experiments in this Example 4 demonstrate that blood glucose level is inversely related to reflection ratio E. The relationship is maintained in the healthy as well as in diabetic blood glucose range (from 87-224 mg/dL) in the data above.

The combined data of Tables 7 and 8 is shown in FIG. 16B.

Based on the data, this relationship can be depicted as the following glucose value formula:

Glucose value=450-350*Ratio

To confirm this finding, the blood glucose of a random subject was estimated. The measured ratio was 1.01. The estimated blood glucose using the disclosed device of FIG. 2 is:

Glucose value=450-350(1.01)=96.5 mg/dl.

This subject's blood glucose was also measured by a blood draw method at the same time, and the results were 99.5 mg/dl. These values are significantly close and clinically similar, which demonstrates that the disclosed device of FIG. 2 can be used for accurate measurement of blood glucose.

Glucose is not a typical chromophore and is in small quantity in blood (60-100 mg/dl in health). In the disclosed device, the electrical signal from photodiodes was amplified about 10,000 times its original value. This increases the sensitivity by about 8,000-fold to about 10,000-fold, and allows detection, as well as any minor changes in the reflectance pattern, resulting due to underlying blood glucose change. Stated another way, the detected value is about 8,000 to about 10,000 times less than the reading displayed by the device of FIG. 2. As one specific example of such an amplification, the ratio is 1.025 mA or 1025 microamps or 1025×1000 nanoamps. The original value is 1025×1000/10,000=102 nanoamps.

### EXAMPLE 5

Using a device disclosed above, including sensors 24′, the reflectance ratio of 4 human subjects was estimated with a detection wavelength of about 1550 nm. Lactate absorbs light at this wavelength and reflects the portion of the light. The sensor 24′ (which is a reverse bias 1550 nm LED, as shown in FIG. 17C) detects this reflected light and converts it into the electrical current. This current is optionally amplified and measured to estimate the reflectance ratio.

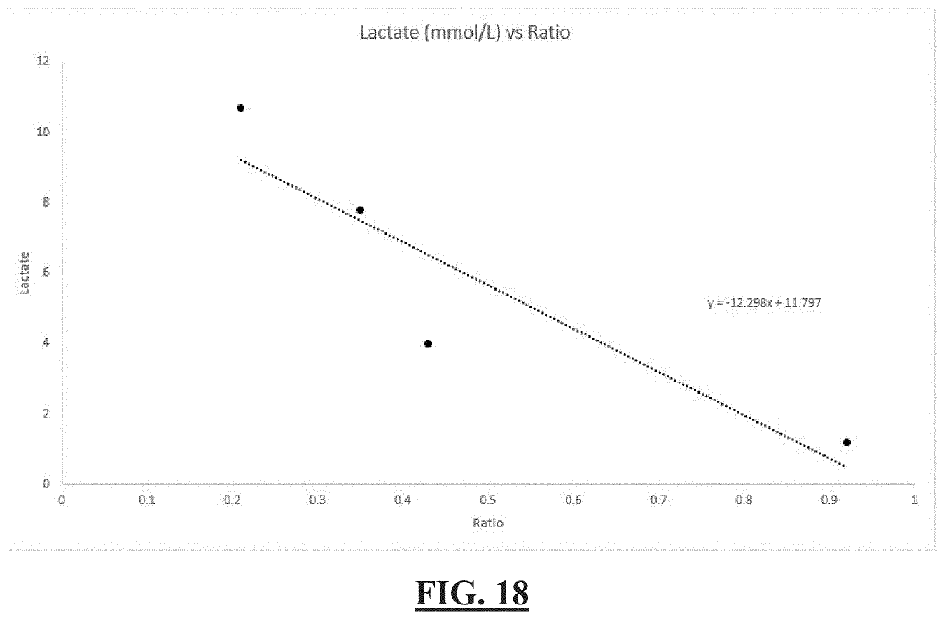

Lactate levels using the disclosed device were measured in 4 subjects. Additionally, lactate levels were detected by the blood draw method at the same time. The results are tabulated below in TABLE 9 and illustrated in FIG. 18.

TABLE 9

| Subject Number | Ratio | Lactate (mmol/L) |
|---|---|---|
| 1 | 0.92 | 1.2 |
| 2 | 0.43 | 4 |
| 3 | 0.21 | 10.7 |
| 4 | 0.35 | 7.8 |

Based on the observations of Table 9 and FIG. 18, this relationship can be depicted as the following lactate value formula:

Lactate value=11.8-12.3 (Ratio)

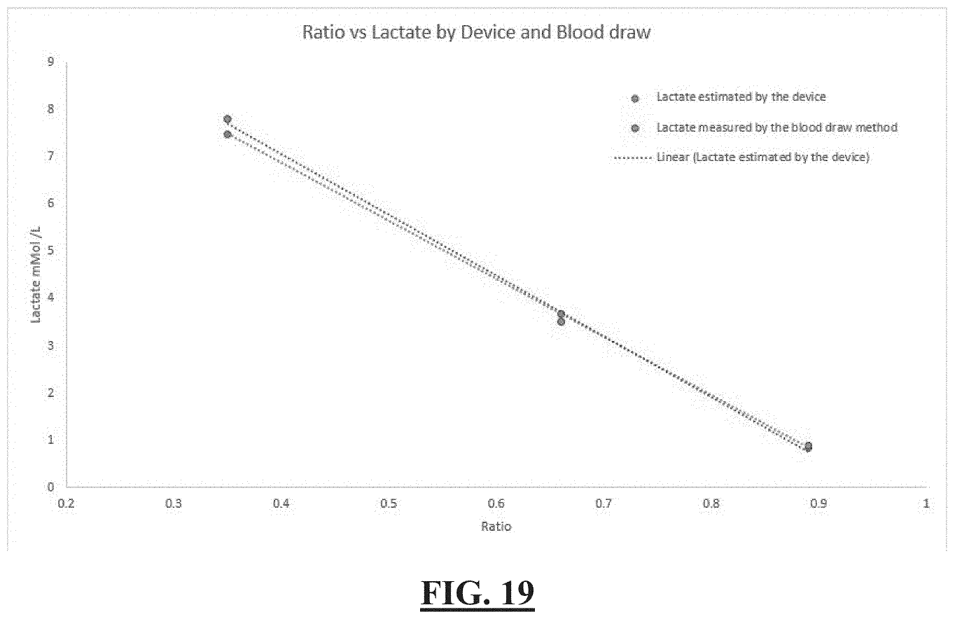

To test this equation, lactate levels (mmol/L) were estimated using the disclosed device and compared with the corresponding values measured by the blood draw. This was done in one subject, before, during and after exercise. Lactate levels are expected to rise after the exercise. These results are shown in Table 10 below and illustrated in FIG. 19.

TABLE 10

| | Ratio | Lactate | Lactate |
|---|---|---|---|
| | measured | estimated | measured by |
| | by the | by the | the blood draw |
| Time | device | device | method |
| Baseline | 0.89 | 0.85 | 0.9 |
| During exercise | 0.66 | 3.68 | 3.5 |
| After exercise | 0.35 | 7.49 | 7.8 |

As seen in Table 10 and in FIG. 19, the disclosed device estimated lactate and the lactate level determined by blood draw are similar. Significantly, the disclosed device is able to detect expected changes during exercise. Thus, it can be used as a non-invasive technique to monitor athletic performance via varying lactate levels.

The described embodiments and examples of the present disclosure are intended to be illustrative rather than restrictive and are not intended to represent every embodiment or example of the present disclosure. While the fundamental novel features of the disclosure as applied to various specific embodiments thereof have been shown, described, and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. A device comprising:
a plurality of light emitting diodes configured to emit light at a wavelength in a range of about 795 nm to about 835 nm towards a surface of a mammal's skin surface.
one or more sensors configured to detect an amount of reflected light in a range of about 795 nm to about 835 nm, wherein the detected, reflected light is the emitted light that passes through at least a portion of the mammal's skin and is at least partially reflected towards the one or more sensors, wherein the one or more sensors are configured to output a signal comprising the amount of the detected, reflected light in the range of about 795 nm to about 835 nm: and
a processor configured to:
send a signal of emitted light at the wavelength in the range of about 795 nm to about 835 nm,
receive the signal of the amount of the detected, reflected light in the range of about 795 nm to about 835 nm,
determine a first ratio of reflected light,
determine a creatinine value by applying the first ratio to a creatinine value formula, and
output the creatinine value.

2. The device of claim 1, wherein the processor is further configured to
send a signal of emitted light at the wavelength in the range of about 805 nm to about 825 nm,
receive the signal of the amount of the detected, reflected light in the range of about 805 nm to about 825 nm.

3. The device of claim 1, wherein the processor is further configured to
send a signal of emitted light at the wavelength in the range of about 815,
receive the signal of the amount of the detected, reflected light in the range of about 815 nm.

4. The device of claim 1, wherein the creatinine value are output to a display that is configured to display text and/or images of the value.

5. The device of claim 4, wherein the display is configured to receive a touch input.

6. A device comprising:
a plurality of light emitting diodes configured to emit light at a wavelength in a range of about 1030 nm to about 1070 nm towards a surface of a mammal's skin surface.
one or more sensors configured to detect an amount of reflected light in a range of about 1030 nm to about 1070 nm, wherein the detected, reflected light is the emitted light that passes through at least a portion of the mammal's skin and is at least partially reflected towards the one or more sensors, wherein the one or more sensors are configured to output a signal comprising the amount of the detected, reflected light in the range of about 1030 nm to about 1070 nm: and
a processor configured to:
send a signal of emitted light at the wavelength in the range of about 1030 nm to about 1070 nm,
receive the signal of the amount of the detected, reflected light in the range of about 1030 nm to about 1070 nm,
determine a first ratio of reflected light,
determine a glucose value by applying the first ratio to a glucose value formula, and
output the glucose value.

7. The device of claim 6, wherein the processor is further configured to
send a signal of emitted light at the wavelength in the range of about 1040 nm to about 1060 nm,
receive the signal of the amount of the detected, reflected light in the range of about 1040 nm to about 1060 nm.

8. The device of claim 6, wherein the processor is further configured to
send a signal of emitted light at the wavelength in the range of about 1050,
receive the signal of the amount of the detected, reflected light in the range of about 1050 nm.

9. The device of claim 6, wherein the glucose value is output to a display that is configured to display text and/or images of the value.

10. The device of claim 9, wherein the display is configured to receive a touch input.

11. A device comprising:
a plurality of light emitting diodes configured to emit light at a wavelength in a range of about 1530 nm to about 1570 nm towards a surface of a mammal's skin surface;
one or more sensors configured to detect an amount of reflected light in a range of about 1530 nm to about 1570 nm, wherein the detected, reflected light is the emitted light that passes through at least a portion of the mammal's skin and is at least partially reflected towards the one or more sensors, wherein the one or more sensors are configured to output a signal comprising the amount of the detected, reflected light in the range of about 1530 nm to about 1570 nm: and
a processor configured to:
send a signal of emitted light at the wavelength in the range of about 1530 nm to about 1570 nm,
receive the signal of the amount of the detected, reflected light in the range of about 1530 nm to about 1570 nm,
determine a first ratio of reflected light,
determine a lactate value by applying the first ratio to a lactate value formula, and
output the lactate value.

12. The device of claim 11, wherein the processor is further configured to
send a signal of emitted light at the wavelength in the range of about 1540 nm to about 1560 nm,
receive the signal of the amount of the detected, reflected light in the range of about 1540 nm to about 1560 nm.

13. The device of claim 11, wherein the processor is further configured to
send a signal of emitted light at the wavelength in the range of about 1550,
receive the signal of the amount of the detected, reflected light in the range of about 1550 nm.

14. The device of claim 11, wherein the lactate value is output to a display that is configured to display text and/or images of the value.

15. The device of claim 14, wherein the display is configured to receive a touch input.
